(19) United States
(12) Patent Application Publication
(10) Pub. No.: US 2025/0213236 A1
(43) Pub. Date: Jul. 3, 2025

(54) INSTRUMENTS AND METHOD FOR FACILITATING NERVE LATERALIZATION & TRANSPOSITION IN ORAL SURGERIES

(71) Applicant: Pedro Barreiros, Porto (PT)

(72) Inventor: Pedro Barreiros, Porto (PT)

(73) Assignee: JESUS & BARREIROS LDA, Porto (PT)

(21) Appl. No.: 18/401,403

(22) Filed: Dec. 30, 2023

(51) Int. Cl. A61B 17/00

(52) U.S. Cl. CPC A61B 17/00234; A61B 2017/00238; A61B 2017/00743; A61B 2017/00831

(57) ABSTRACT

This invention encompasses an innovative set of instruments (BA.P.LAT 1, BA.P.LAT 2, BA.P.LAT 3) and an associated surgical method designed to simplify nerve lateralization & transposition in oral surgery. The instruments facilitate safe handling, mobilization, and protection of the inferior alveolar nerve, enabling safer and more effective placement of dental implants. This invention represents a significant advancement in oral surgery, making the procedure safer, more efficient, and accessible to a wider range of surgeons.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

This invention pertains to the field of Oral Surgery, specifically to tools and techniques facilitating the process of nerve lateralization and transposition, thus simplifying surgical procedures involving the inferior alveolar nerve (IAN).

### Background of Invention

Surgical techniques involving the IAN, such as implant dentistry and oral surgery, often face challenges due to the intricacies of nerve handling, particularly when limited by a deficient alveolar ridge. The IAN runs through the mandibular canal and is midway between the buccal and lingual cortical plates. Its displacement is crucial for the safe placement of dental implants without damaging the nerve or requiring bone augmentation. Conventional instruments and methods have a degree of risk, including potential nerve damage and inadequate space for implant placement.

Inferior alveolar nerve (IAN) lateralization is a complex and critical procedure in oral surgery and implant dentistry, often employed when there is insufficient alveolar bone for conventional implant placement. The procedure, while effective, presents certain challenges due to the delicate nature of handling the IAN, the potential for nerve damage, and the complications arising from a deficient alveolar ridge.

In the traditional approach to IAN lateralization, a mucoperiosteal flap is created to expose the underlying bone. Then, a lateral window is cut into the bone using specialized instruments like a piezosurgery motor. This motor is typically used to cut medular bone around the nerve without damaging it, thus isolating the IAN. Despite careful handling, the risk of nerve damage is a persistent concern. Existing tools often fall short in preventing damage to the nerve and surrounding tissues. In particular, the task of mobilizing and retracting the IAN to a lateral position to create room for dental implant placement is especially delicate, with even minor slippage potentially causing serious nerve injury.

Further challenges in the existing method of surgery involve the space constraint for implant placement. The traditional lateralization procedure does not ensure optimal space availability for implant insertion due to variability in the individual's oral anatomy and the restrictions posed by the nerve's positioning. These limitations may necessitate bone augmentation—an additional, invasive procedure with its own set of complications and risks.

Therefore, despite the utility of IAN lateralization in overcoming limitations of deficient alveolar bone, the associated risks and complications call for an improved methodology. A need exists for specialized instruments designed to safely handle and manipulate the IAN, thereby reducing the risks associated with conventional procedures, optimizing the available space for implant placement, and rendering the procedure safer and more efficient for a broader range of surgeons.

### BRIEF SUMMARY OF THE INVENTION

The present invention provides an innovative set of instruments, known as BA.P.LAT 1, BA.P.LAT 2, and BA.P.LAT 3, developed to assist in the mobilization and protection of the IAN during oral surgeries, particularly nerve lateralization and transposition. These instruments offer better handling and protection of the IAN, creating a safe and efficient surgical environment. Additionally, the invention includes a method for using these instruments, effectively addressing the challenges of conventional techniques and making the procedure safer and more accessible to surgeons.

### DESCRIPTION OF THE DRAWINGS

The following descriptions of the drawings are provided to illustrate the three instruments in the invention, namely BA.P.LAT 1, BA.P.LAT 2, and BA.P.LAT 3, and to further elucidate their design and functionality.

FIG. 1 is a perspective view of the three instruments of the proposed invention.

FIG. 2 is a left side view of the three instruments of the proposed invention.

FIG. 3 is a right side view of the three instruments of the proposed invention.

FIG. 4 is a rear view of the three instruments of the proposed invention.

FIG. 5 is a front view of the three instruments of the proposed invention.

FIG. 6 is a top view of the three instruments of the proposed invention.

FIG. 7 is a bottom view of the three instruments of the proposed invention.

In conclusion, these views together provide a comprehensive visualization of the unique design elements of the first instrument BA.P.LAT 1, second instrument BA.P.LAT 2, and third instrument BA.P.LAT 3, and their role in enhancing the safety and efficacy of oral surgical procedures involving the IAN.

### REFERENCE NUMERALS IN THE DRAWINGS

For a more complete understanding of the present invention parts, reference is now made to the following descriptions:
100. The first instrument BA.P.LAT 1.
102. The first instrument BA.P.LAT 1 tip.
104. The first Instrument BA.P.LAT 1 handle.
106. The second instrument BA.P.LAT 2.
108. The second instrument BA.P.LAT 2 tip.
110. The second instrument BA.P.LAT 2 handle.
112. The third instrument BA.P.LAT 3.
114. The third instrument BA.P.LAT 3 tip.
116. The third instrument BA.P.LAT 2 handle.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

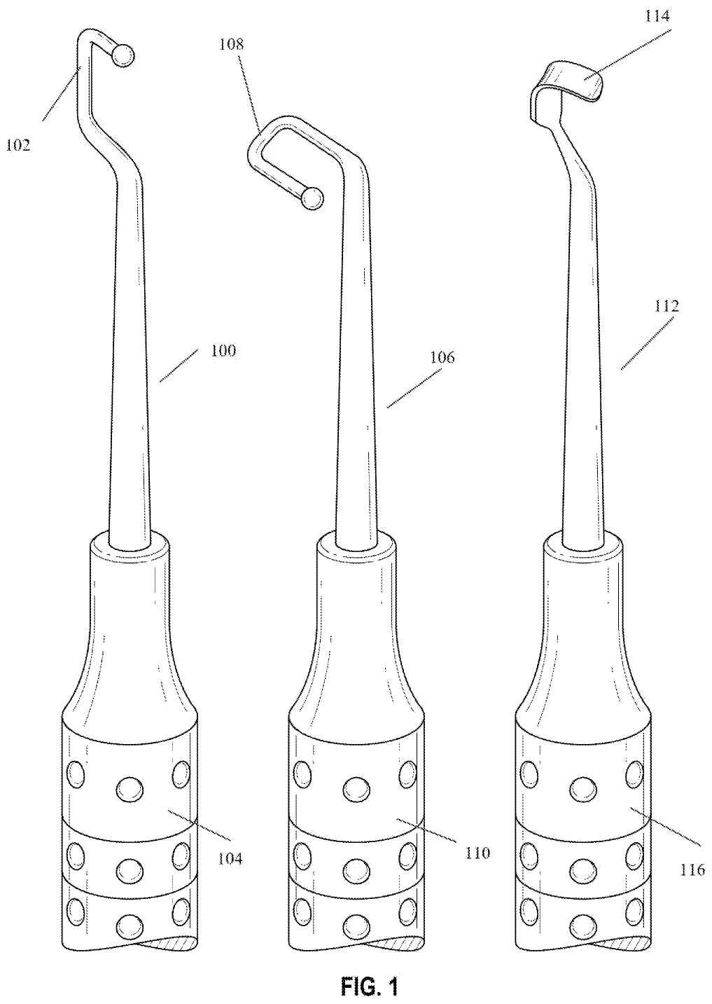

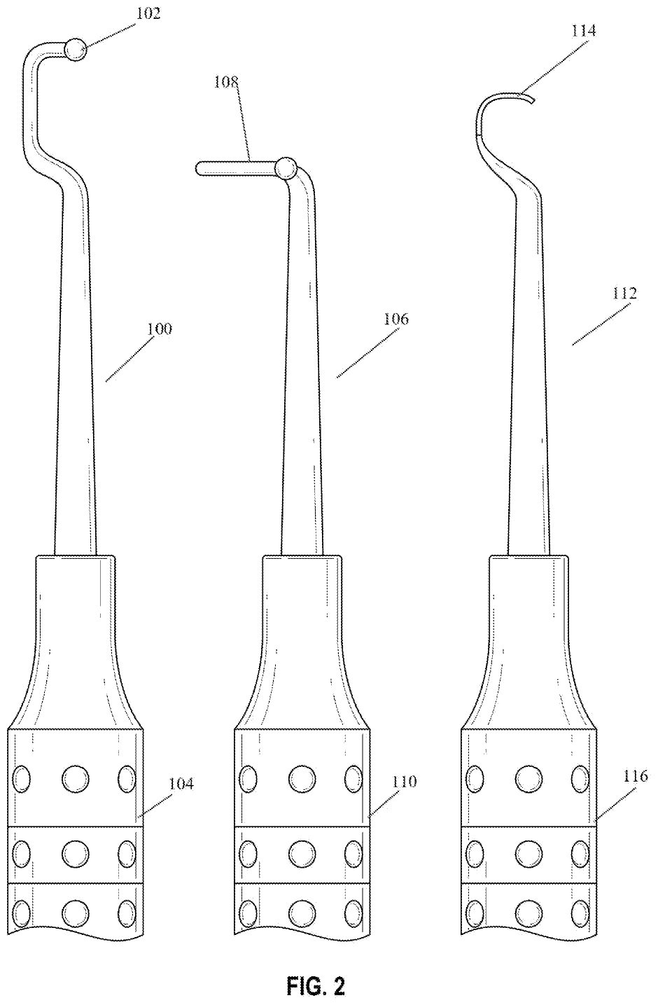

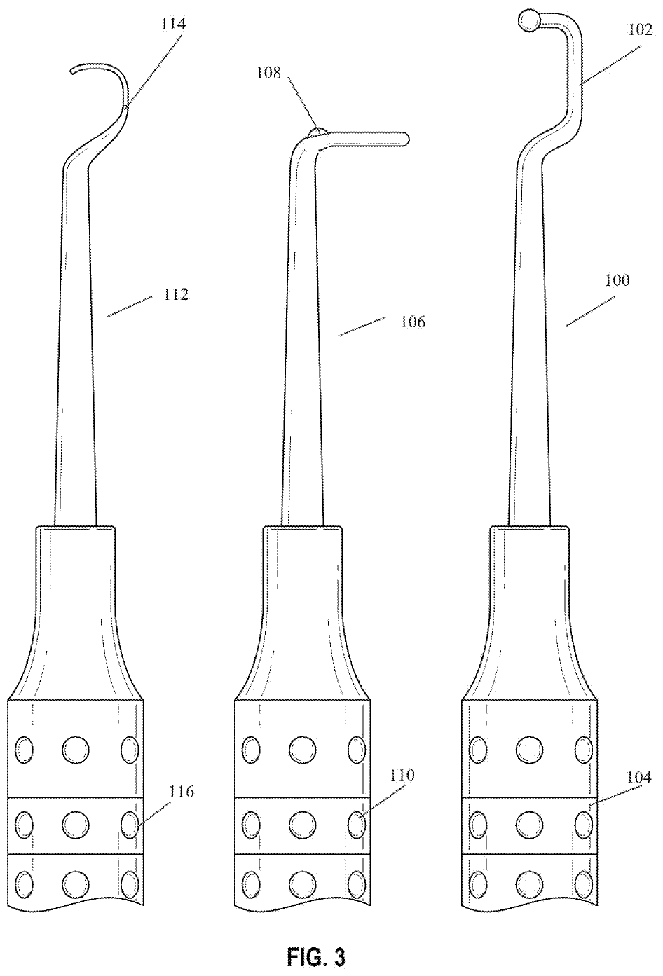

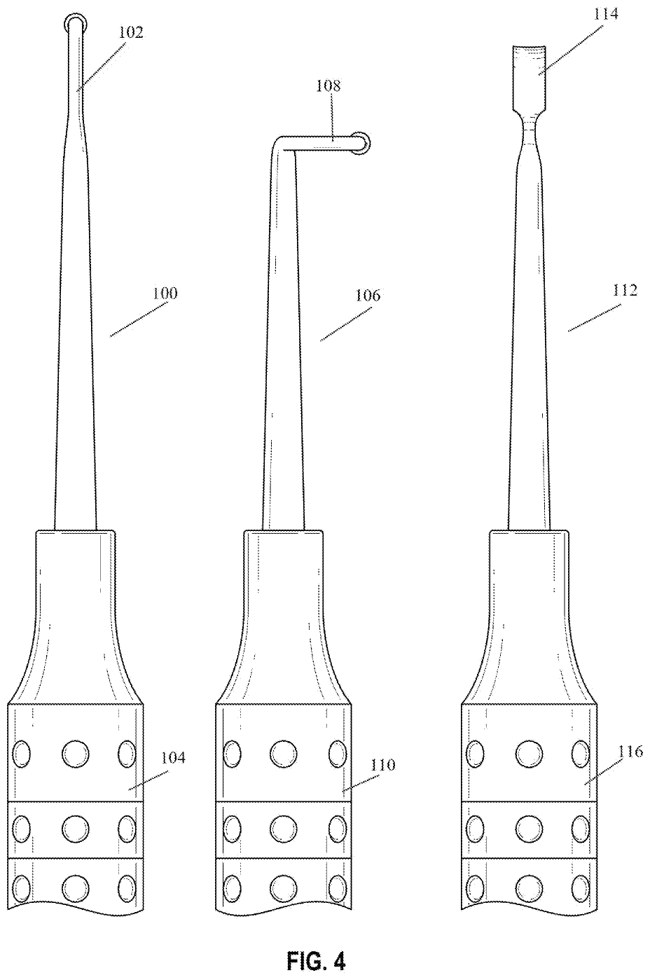

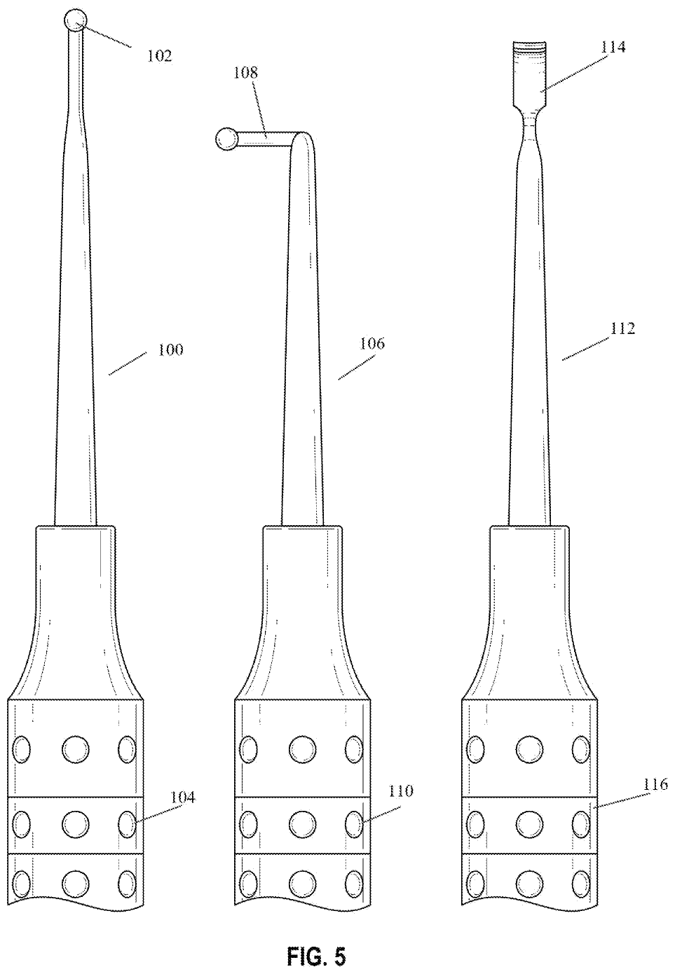

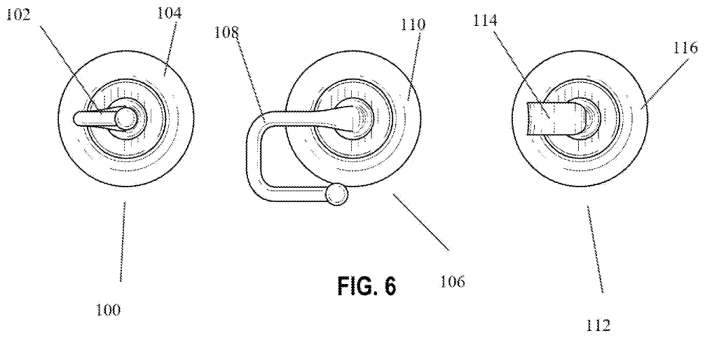

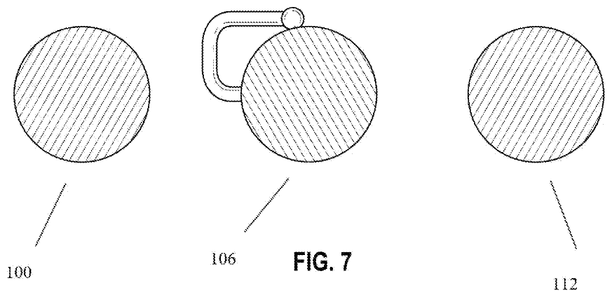

Referring to FIG. 1, FIG. 2, FIG. 3, FIG. 4, FIG. 5, FIG. 6, and FIG. 7, the present invention revolves around a trio of instruments specifically curated to facilitate nerve lateralization and transposition surgery, namely BA.P.LAT 1 (100), BA.P.LAT 2 (106), and BA.P.LAT 3 (112). BA.P.LAT 1 (100) is composed of a tip (102) and a handle (104). The tip (102) of BA.P.LAT 1 (100) is meticulously engineered to safely and delicately manipulate the inferior alveolar nerve (IAN) without straining or causing harm. This is achieved through its specific size and shape, which permits a small, but sufficient cortical window to be opened during surgery. The handle (104) of BA.P.LAT 1 (100) is ergonomically constructed to allow the surgeon an optimal grip, lending precision and control during the surgical process.

BA.P.LAT 2 (106) also possesses a tip (108) and a handle (110), similar to BA.P.LAT 1 (100). However, its uniqueness lies in its utility in a horizontal approach, necessary in instances of difficult or different intraoral access. BA.P.LAT 2's tip (108) is expertly crafted to laterally move and handle the IAN. Simultaneously, the handle (110) is designed to provide a firm and comfortable grip to the surgeon, ensuring the tool is maneuvered with care and precision.

Lastly, BA.P.LAT 3 (112) is designed with a specialized tip (114) and handle (116). The tip (114) of BA.P.LAT 3 (112) plays an important role in protecting the IAN during surgical drill preparation and implant placement. It is built to shield the IAN from potential harm such as cuts, curls, or lacerations. The handle (116) of BA.P.LAT 3 (112) enables the surgeon to maintain a strong grip and control during these pivotal steps, enhancing surgical precision.

Now let us delve into how these instruments are deployed in the nerve lateralization and transposition surgery. The surgery commences with the opening of a cortical window. The surgeon, utilizing the tip (102) and handle (104) of BA.P.LAT 1 (100), gently manipulates and moves the IAN without causing tension or risk of damage.

For cases where horizontal approach is required, BA.P.LAT 2 (106) comes into play. With the aid of the tip (108) and the handle (110), the surgeon can achieve the same delicate handling and movement of the IAN as BA.P.LAT 1 (100), but in a different orientation, which could be crucial due to the variable oral anatomy of patients. As the surgery progresses to the stage of surgical drill preparation and implant placement, BA.P.LAT 3 (112) is employed. With its protective tip (114), BA.P.LAT 3 (112) safeguards the IAN, while the handle (116) allows for precision control by the surgeon.

In conclusion, the trio of instruments-BA.P.LAT 1 (100), BA.P.LAT 2 (106), and BA.P.LAT 3 (112)—brought forth by this invention significantly enhances the safety, control, and accessibility of nerve lateralization and transposition procedures in oral surgery. Each tool, with its specially designed tip and handle, ensures optimal protection for the IAN, superior surgical precision, and expanded accessibility of the procedure to a larger cadre of surgeons.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A set of medical instruments for facilitating nerve lateralization & transposition in oral surgeries, comprising:
the first instrument BA.P.LAT 1, a tool designed to handle, move, and lateralize the inferior alveolar nerve without tension or risk of damage;
the second instrument BA.P.LAT 2, a tool similar to the first instrument BA.P.LAT 1 but designed for use in a horizontal approach when necessary; and,
the third instrument BA.P.LAT 3, a tool used to protect the inferior alveolar nerve during surgical drill preparation and dental implant placement, thereby preventing damage to the neurovascular bundle.

2. The set of medical instruments of claim 1, where all instruments, including the first instrument BA.P.LAT 1, the second instrument BA.P.LAT 2, and the third instrument BA.P.LAT 3 are made of medical-grade, sterilizable steel.

3. The set of medical instruments of claim 1, wherein each instrument, including the first instrument BA.P.LAT 1, the second instrument BA.P.LAT 2, and the third instrument BA.P.LAT 3 is uniquely designed to safely mobilize the inferior alveolar nerve to a new temporary position to facilitate the placement of dental implants.

4. A method for facilitating nerve lateralization and transposition in oral surgeries using the set of medical instruments of claim 1, the method comprising:
making an incision along the lower jaw's gum line;
creating a mucoperiosteal flap;
cutting a lateral bone window to access the inferior alveolar nerve;
mobilizing and retracting the nerve to a lateral position using the first instrument BA.P.LAT 1 or the second instrument BA.P.LAT 2;
preparing the bone for implant placement;
placing the implants;
positioning the nerve near the implants, with the nerve protected by the third instrument BA.P.LAT 3;
replacing and stabilizing the cortical bone windows using bone plates and screws; and,
closing the surgical site.

5. The method of claim 4, wherein the first instrument BA.P.LAT 1 is used for typical access, the second instrument BA.P.LAT 2 is used for a horizontal approach, and the third instrument BA.P.LAT 3 is used for protection during surgical drill preparation and implant placement.

6. The method of claim 4, wherein the use of the first instrument BA.P.LAT 1, the second instrument BA.P.LAT 2, and the third instrument BA.P.LAT 3 minimizes the risk of nerve damage and maximizes the space available for implant placement.

7. The method of claim 4, wherein each step is performed under local anesthesia to reduce patient discomfort.

8. The method of claim 4, wherein the technique using the first instrument BA.P.LAT 1, the second instrument BA.P.LAT 2, and the third instrument BA.P.LAT 3 facilitates the procedure of nerve lateralization and transposition, making it more accessible and safer for a broader range of surgeons.
